(19) United States
(12) Patent Application Publication
(10) Pub. No.: US 2021/0016258 A1
(43) Pub. Date: Jan. 21, 2021

(54) METHOD FOR PRODUCING BETA-COBALT MOLYBDENUM OXIDE CATALYST HAVING ENHANCED SELECTIVITY FOR THE PRODUCTION OF C3-C4 ALCOHOLS AND CATALYST OBTAINED THEREBY

(71) Applicant: SABIC Global Technologies B.V., Bergen op Zoom (NL)

(72) Inventor: Muhammad H. HAIDER, Riyadh (SA)

(73) Assignee: SABIC Global Technologies B.V., Bergen op Zoom (NL)

(21) Appl. No.: 15/733,706

(22) PCT Filed: May 7, 2019
(86) PCT No.: PCT/IB2019/053736

§ 371 Date: Sep. 30, 2020

Related U.S. Application Data
(60) Provisional application No. 62/670,197, filed on May 11, 2018.

(51) Int. Cl. B01J 23/882; C07C 29/156; B01J 37/00

(52) U.S. Cl. CPC B01J 23/882; B01J 2523/00; B01J 37/0009; C07C 29/156

(57) ABSTRACT

Methods for producing cobalt/molybdenum catalysts having enhanced selectivity for the production of C₃-C₄ alcohols. The catalyst production methods allow for the selective production of beta-phase catalysts over alpha-phase catalysts. The catalyst is a calcined composition comprising: β-CoxMoyOz, wherein x ranges from 0.5 to 2.0, y ranges from 0.5 to 2.0, and z ranges from 3.5 to 4.5, wherein said calcined composition is essentially free of catalytically-active amounts of beta-molybdenum carbide (β-Mo2 C), and wherein said calcined composition is essentially free of catalyst-promoting amounts of an alkaline metal promoter or alkaline earth metal promoter.

### CROSS REFERENCE TO RELATED APPLICATIONS

This application claims the benefit of priority to U.S. Provisional Application No. 62/670,197, filed May 11, 2018, the entire contents of which are incorporated herein by reference in their entirety.

### FIELD OF INVENTION

The present invention generally relates to the production of catalysts that selectively catalyze the production of C₃ and C₄ alcohols from synthesis gas.

### BACKGROUND OF THE INVENTION

The interest in converting synthesis gas (syngas) to alcohols is growing rapidly. Syngas, a mixture of carbon monoxide and hydrogen, with some carbon dioxide in some cases, can be obtained from various carbon-containing sources such as coal, natural gas, biomass, and as a by-product of various chemical production processes.

A variety of products, including paraffins, alcohols, olefins, and other chemicals can be obtained from the catalytic conversion of syngas. One significant syngas conversion route is via lower alcohol, i.e., C₃-C₄ alcohol, synthesis. Butanol is an important industrial chemical with a wide range of applications. It can be used as a motor fuel, particularly in combination with gasoline to which it can be added in all proportions. Propanol and butanol can be converted into the polymer precursors propylene and butylene, respectively, through a dehydration reaction. Butanol can be converted into butadiene by successive dehydration and dehydrogenation reactions. Isobutanol can also be used a precursor to isobutylene and Methyl Tertiary Butyl Ether (MTBE).

As of late, research in olefin production from syngas over cobalt/molybdenum catalysts has increased, as lower olefins have increased in utility. This is due to ever-increasing demand of C₂-C₄ olefins globally in the manufacturing of many plastic-based products. The production of lower olefins using cobalt/molybdenum catalysts is not well-established, as the currently-available cobalt/molybdenum catalysts are more selective to longer chain products. There exists a need in the industry for the production of cobalt/molybdenum catalysts having improved lower alcohol selectivity. The lower alcohols produced by these methods can act as precursors for C₃ and C₄ olefins.

### BRIEF SUMMARY OF THE INVENTION

A method has been discovered for production of propanol and butanol, which upon dehydration can give very clean high yields of propylene and butylene. The method employs a cobalt/molybdenum catalyst having a β-phase crystal structure. A comparison of the β-phase cobalt/molybdenum catalyst with α-phase cobalt/molybdenum catalyst shows that the yield of C₃-C₄ alcohols is higher with the β-phase catalyst than the α-phase catalyst.

In some aspects, the disclosure provides a calcined composition comprising β-CoₓMo_{y}O_{z}, wherein x ranges from 0.5 to 2.0, y ranges from 0.5 to 2.0, and z ranges from 3.5 to 4.5. In some aspects, the calcined composition is essentially free of catalytically-active amounts of beta-molybdenum carbide (β-Mo₂C). In some embodiments, the calcined composition is essentially free of catalyst-promoting amounts of an alkaline metal promoter or alkaline earth metal promoter. In some embodiments, the calcined composition is essentially free of a carbon support.

In some embodiments, a process for the conversion of a synthesis gas stream into a product stream comprising C₃-C₄ alcohols is provided. The method comprises exposing a synthesis gas stream to a calcined composition under conditions suitable to convert at least 10% of the synthesis gas stream with at least 35% selectivity for C₃-C₄ alcohols, wherein said calcined composition comprises β—CoₓMo_{y}O_{z}, with x ranging from 0.5 to 2.0, y ranging from 0.5 to 2.0, and z ranging from 3.5 to 4.5. In some aspects, the calcined composition is essentially free of catalytically-active amounts of beta-molybdenum carbide (β-Mo₂C). In additional aspects, the calcined composition is essentially free of catalyst-promoting amounts of an alkaline metal promoter or alkaline earth metal promoter.

In further embodiments, a method for making a β-phase catalyst capable of producing C₃-C₄ alcohols from a synthesis gas stream with at least 25% conversion and at least 50% selectivity is provided. In some aspects, the method comprises the steps of preparing a solution comprising a cobalt salt and a molybdenum salt and collecting a precipitate from the solution; drying the precipitate to give a dried precipitate comprising one or more hydrates of cobalt molybdenum oxide; pelleting the dried precipitate to produce pellets; and calcining the pellets to generate the β-phase catalyst. In specific aspects, the pellets are not subjected to mechanical deformation subsequent to calcination.

The following includes definitions of various terms and phrases used throughout this specification. The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. “Essentially free” is defined as having no more than about 0.1% of a component. For example, a calcined composition being essentially free of catalytically-active amounts of beta-molybdenum carbide (β-Mo₂C) has no more than about 0.1% of beta-molybdenum carbide, by weight.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 is graph depicting CO conversion and product selectivity profile for batch 1 of powdered β-CoMoO₄.

FIG. 2 is graph depicting CO conversion and product selectivity profile for batch 2 of powdered β-CoMoO₄.

FIG. 3 is a graph depicting CO conversion and product selectivity profile for α-CoMoO₄ in powdered form.

FIG. 4 is a graph depicting CO conversion and product selectivity profile for α-CoMoO₄ in pellet form.

FIG. 5 is a graph depicting CO conversion and product selectivity profile for batch 1 of β-CoMoO₄ in pellet form.

FIG. 6 is a graph depicting CO conversion and product selectivity profile for batch 2 of β-CoMoO₄ in pellet form.

FIG. 7 is a graph depicting CO conversion and product selectivity profile for batch 3 of β-CoMoO₄ in pellet form.

### DETAILED DESCRIPTION OF THE INVENTION

Cobalt/molybdenum oxide catalysts of the formula CoMoO₄ can exist in α- or β-crystal forms. Although the two forms may have similar stoichiometries, their distinct crystal structures play a role in their respective catalytic activities. A method has been discovered for the preparation of a cobalt/molybdenum catalyst that maintains a β-phase crystal structure during work-up and processing. The β-phase catalyst exhibits improved syngas conversion and butanol selectivity.

Through investigating cobalt/molybdenum catalyst activities, the inventor has discovered that conventional catalyst processing, specifically, post-calcination grinding or pelletization, induces the phase change of β-CoMoO₄ to α-CoMoO₄. Without wishing to be bound by theory, it is believed that the energy transmitted to the calcined catalyst by grinding or pelletization enables the conversion of β-crystallites to α-crystallites. The two crystal forms can be visually differentiated by their colors; β-CoMoO₄ is purple, whereas α-CoMoO₄ is green. More importantly, the two crystal forms can be experimentally distinguished by their distinct catalytic activities.

The inventor has developed a strategy that preserves the improved-activity β-phase before reduction in situ. Preparing catalyst powder or pellets before calcination (when the catalyst is in the hydrated form of β-CoMoO₄) ensures the catalyst remains in the β-form and provides high selectivity towards C₃-C₄ alcohols at a conversion of approximately 30%. In a further aspect, the alcohols produced by this process can be dehydrated into the corresponding olefins. Dehydration can be performed at a temperature above alcohol boiling points in the presence of an acid-type catalyst, e.g., cesium-doped silicotungstic acid supported on alumina.

In some aspects, the disclosure provides a calcined composition comprising β-CoₓMo_{y}O_{z}, wherein x ranges from 0.5 to 2.0, y ranges from 0.5 to 2.0, and z ranges from 3.5 to 4.5. In some aspects, the calcined composition is essentially free of catalytically-active amounts of beta-molybdenum carbide (β-Mo₂C). In some embodiments, the calcined composition is essentially free of catalyst-promoting amounts of an alkaline metal promoter or alkaline earth metal promoter.

In some aspects, the composition exhibits a synthesis gas conversion of at least 10%, under suitable reaction conditions. In preferred aspects, the composition exhibits a synthesis gas conversion of at least 25% under suitable reaction conditions. In some embodiments, the composition exhibits a cumulative C₃-C₄ alcohol selectivity of at least 35% under suitable reaction conditions. In preferred aspects, the composition exhibits a cumulative C₃-C₄ alcohol selectivity of at least 50% under suitable reaction conditions. In some embodiments, suitable reaction conditions include a reactor pressure ranging from 50 to 100 bar, preferably from 60 to 90 bar, more preferably from 70 to 80 bar. In some aspects, suitable reaction conditions include a reactor temperature ranging from 150 to 450° C., preferably from 200 to 400° C., more preferably from 250 to 350° C. In some embodiments, suitable reaction conditions include a synthesis gas CO:H₂ ratio ranging from 0.8:1 to 1.2:1, preferably 1:1. An inert gas, such as nitrogen, may be provided with the synthesis gas in an amount ranging from 1 to 20%, based on the total amount of CO and H₂. In some embodiments, the calcined composition comprises β-CoₓMo_{y}O_{z}, where x ranges from 0.9 to 1.1, y ranges from 0.9 to 1.1, and z ranges from 3.9 to 4.1.

In some embodiments, a process for the conversion of a synthesis gas stream into a product stream comprising C₃-C₄ alcohols is provided. The method comprises exposing a synthesis gas stream to a calcined composition under conditions suitable to convert at least 10% of the synthesis gas stream with at least 35% selectivity for C₃-C₄ alcohols, wherein said calcined composition comprises β-CoₓMo_{y}O_{z}, with x ranging from 0.5 to 2.0, y ranging from 0.5 to 2.0, and z ranging from 3.5 to 4.5. In some aspects, the calcined composition is essentially free of catalytically-active amounts of beta-molybdenum carbide (β-Mo₂C). In additional aspects, the calcined composition is essentially free of catalyst-promoting amounts of an alkaline metal promoter or alkaline earth metal promoter. In some embodiments, the calcined composition comprises β-CoₓMo_{y}O_{z}, where x ranges from 0.9 to 1.1, y ranges from 0.9 to 1.1, and z ranges from 3.9 to 4.1.

In some aspects, the process for the conversion of a synthesis gas stream into a product stream comprising C₃-C₄ alcohols comprises a reactor pressure ranging from 50 to 100 bar, preferably from 60 to 90 bar, more preferably from 70 to 80 bar. In some embodiments, the process for the conversion of a synthesis gas stream into a product stream comprising C₃-C₄ alcohols comprises a reactor temperature ranging from 150 to 450° C., preferably from 200 to 400° C., more preferably from 250 to 350° C. In some embodiments, the process for the conversion of a synthesis gas stream into a product stream comprising C₃-C₄ alcohols a synthesis gas CO:H₂ ratio ranging from 0.8:1 to 1.2:1, preferably 1:1. An inert gas, such as nitrogen, may be provided with the synthesis gas in an amount ranging from 1 to 20%, based on the total amount of CO and H₂.

In further embodiments, a method for making a β-phase catalyst capable of producing C₃-C₄ alcohols from a synthesis gas stream with at least 25% conversion and at least 50% selectivity is provided. In some aspects, the method comprises the steps of preparing a solution comprising a cobalt salt and a molybdenum salt and collecting a precipitate from the solution; drying the precipitate to give a dried precipitate comprising one or more hydrates of cobalt molybdenum oxide; pelleting the dried precipitate to produce pellets; and calcining the pellets to generate the β-phase catalyst. In specific aspects, the pellets are not subjected to mechanical deformation subsequent to calcination. In a preferred embodiment, the cobalt salt is cobalt acetate and the molybdenum salt is ammonium heptamolybdate. In some embodiments, the solution comprises a binary solvent, preferably ethanol and water, more preferably from 10 to 30% ethanol and from 70 to 90% water, even more preferably 20% ethanol and 80% water, vol:vol. In some embodiments, the precipitate is dried at a temperature ranging from 70 to 150° C., preferably from 90 to 130° C., more preferably from 100 to 120° C. In some aspects, the precipitate is dried for a period of time ranging from 4 to 8 hours, preferably from 5 to 7 hours. In some embodiments, the pellets are calcined at a temperature ranging from 300 to 700° C., preferably from 400 to 600° C., more preferably from 450 to 550° C. In some aspects, the pellets are calcined for a period of time ranging from 2 to 6 hours, preferably from 3 to 5 hours, more preferably from 2.5 to 3.5 hours. In some aspects, the pellets are calcined under an ambient air environment. Ambient air is defined as atmospheric air present at the calcination unit. In further embodiments, the pellets are calcined under oxygen, nitrogen, helium, or a combination thereof.

### EXAMPLES

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example 1

### β-CoMo Powder Preparation

Separate solutions (each in 100 ml of a binary solvent; 80% H₂O, 20% EtOH) of cobalt acetate (12.45 g) and ammonium heptamolybdate (8.45 g) were heated to 65° C. to dissolve the salts. The molybdenum solution was heated at 65° C. while stirring, and the cobalt solution was added dropwise using a separatory funnel. The combined solution was aged for 2 h. The solution was then filtered without washing and the dark purple precipitate was dried in an oven (110° C.) for 6 h. The dried catalyst precursor was ground to a powder then calcined (500° C., static air, 10° C./min heating rate, 4 h). The purple color was maintained after calcination. The calcined catalyst (6 ml volume comprising 3 ml catalyst and 3 ml SiC) was then reduced in situ (16 h, H₂, 50 ml/min, 350° C., 1° C. min⁻¹). Two batches, batch 1 (B1) and batch 2 (B2) were than tested to asses reproducibility.

### Example 2

### α-CoMoO₄ Powder and Pellet Preparation

Separate solutions (each in 100 ml of a binary solvent; 80% H₂O, 20% EtOH) of cobalt acetate (12.45 g) and ammonium heptamolybdate (8.45 g) were heated to 65° C. to dissolve the salts. The molybdenum solution was heated at 65° C. while stirring, and the cobalt solution was added dropwise using a separatory funnel. The combined solution was aged for 2 h. The solution was then filtered without washing and the dark purple precipitate was dried in an oven (110° C.) for 6 h. The dried catalyst precursor was ground to a powder then calcined (500° C., static air, 10° C./min, 4 h). The powder was then ground. Post-calcination grinding induced a phase change from β-CoMoO₄ (purple) to α-CoMoO₄ (green). The color and phase change were observed before loading the green α-CoMoO₄ into the reactor. An in situ pre-reduction H₂ step was performed before syngas testing. Both powder and pellets (made at 10 tons pressure) were used.

### Example 3

### β-CoMo Pellet Preparation

In order to confirm that the catalyst prepared in Example 1 is stable in pelleted form and does not change phase upon pelleting, a pelleted version of the Example 1 catalyst (Example 3) was prepared. After preparing the Example 1 catalyst powder described above, the powder was then pelleted (10 ton pressure) then calcined (500° C., static air, 10° C./min, 4 h) to give the final stable pelleted β-CoMoO₄ catalyst. Preparing the catalyst pellets before calcination (when catalyst exists as hydrated form of the β-CoMoO₄) ensured that the catalyst remained in the β-form.

### Catalyst Activity/Selectivity Evaluation

The catalysts produced in Examples 1-3 were evaluated for the activity and selectivity, as well as short- and long-term stabilities. Prior to activity measurement, all of the catalysts were subjected to a reductive activation procedure (H₂, 100 ml/min, 350° C., 1° C./min, 16 h). Catalyst evaluation was carried out in a high-throughput, fixed-bed flow reactor setup housed in temperature-controlled system fitted with regulators to maintain pressure during reactions. The products of the reactions were analyzed through online GC analysis. The evaluation was carried out under the following conditions unless otherwise indicated: 75 bar, 300° C., 1° C./min, 48 h stabilization, 100 ml/min, 50% SiC mix. The mass balances of the reactions were calculated to be 95±5%.

### Results and Discussion

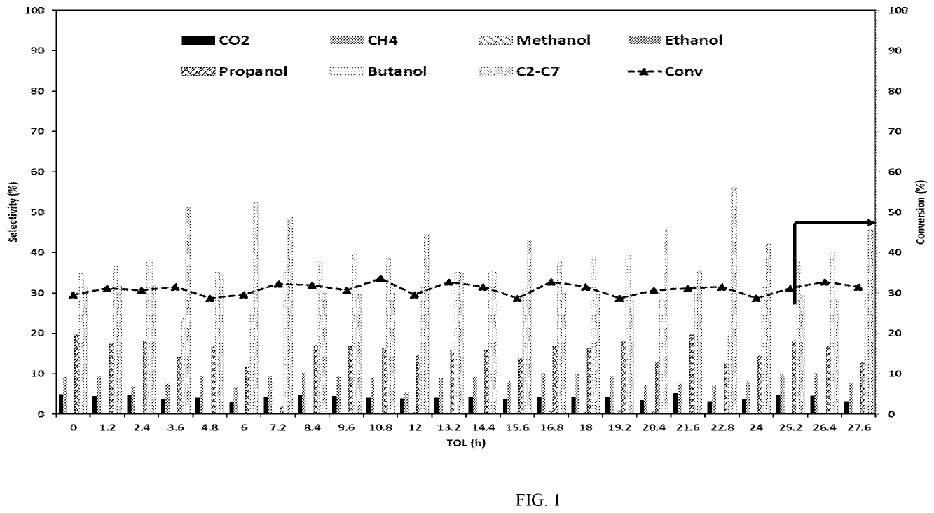

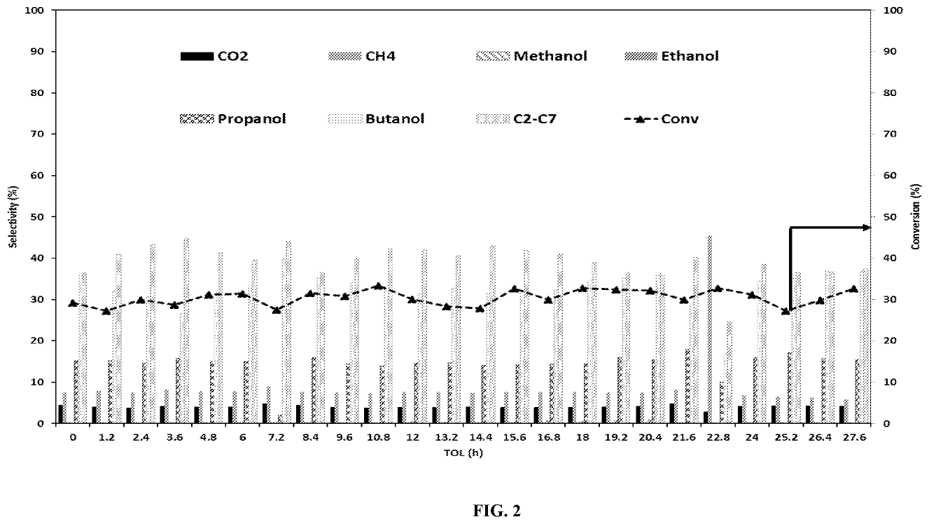

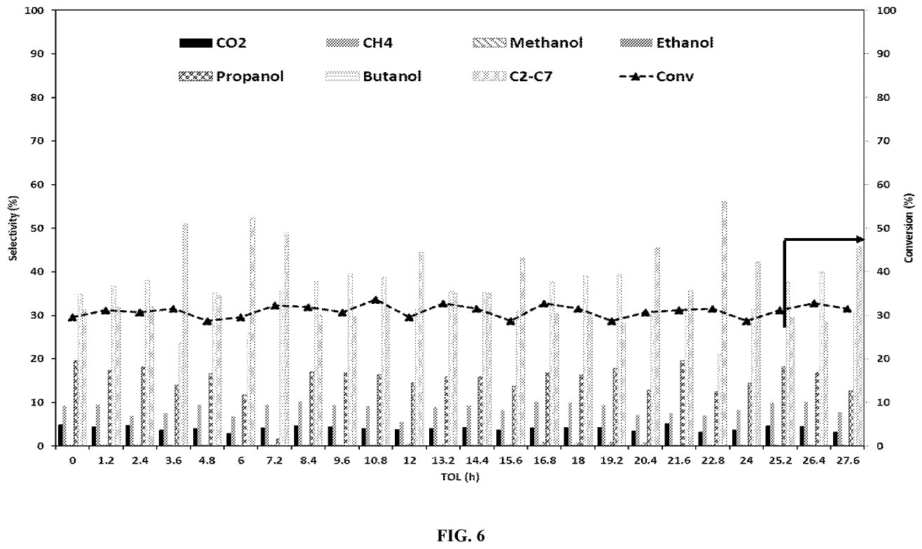

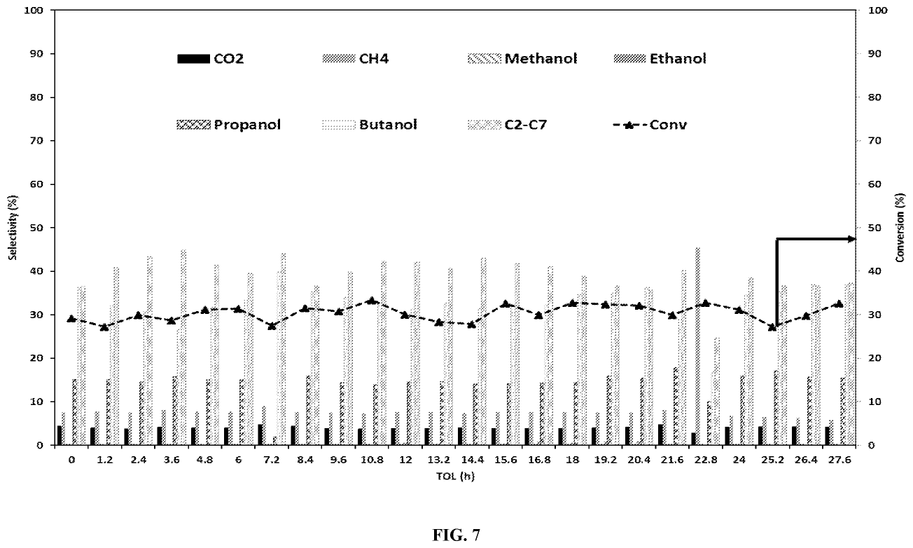

Catalyst testing results are depicted in FIGS. 1-7. FIGS. 1-2 provide results for two catalyst batches prepared in powder form without pelleting, i.e., the β-phase. Cumulative selectivity towards C₃-C₄ alcohols was in the range of 50-60%, with approximately 30% conversion.

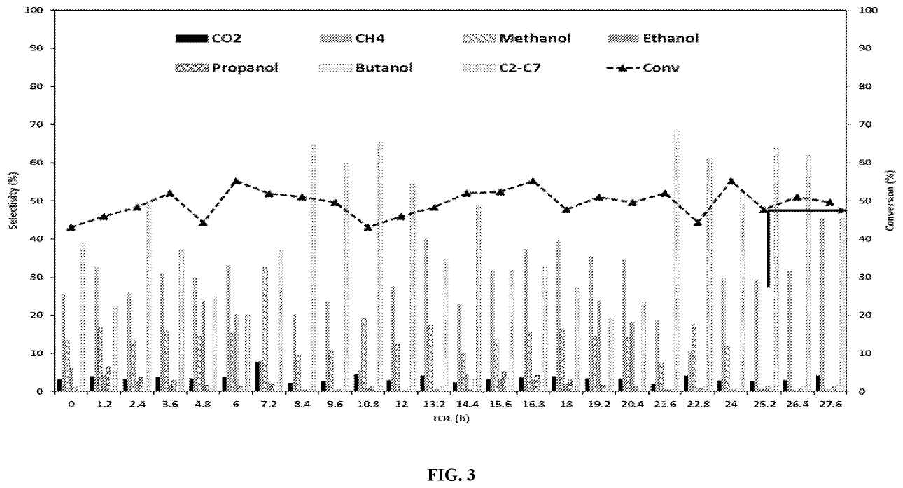

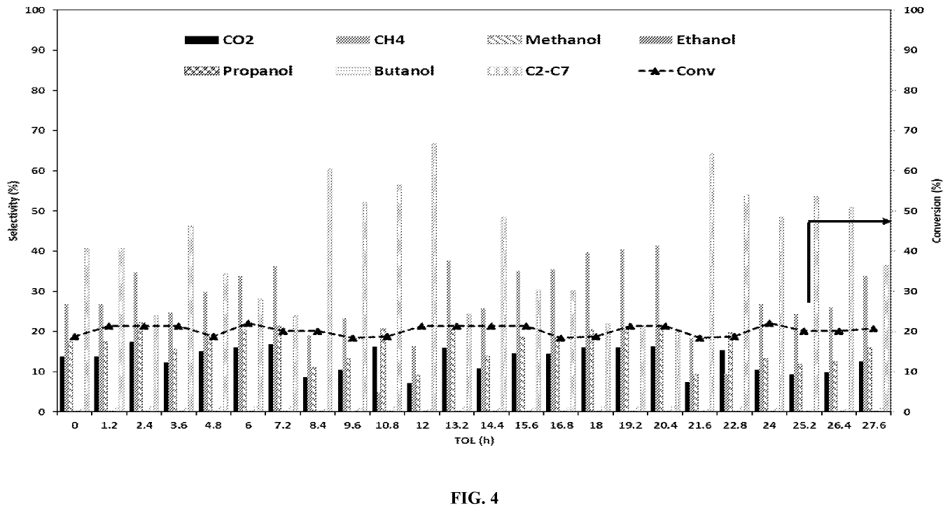

When the catalyst is pelleted/ground post-calcination, the product distribution changes, with methane, methanol, and other hydrocarbons observed as major products (FIGS. 3-4). The distinct product distribution was attributed to the α-CoMoO₄ phase, which was green in color. The results demonstrate that the β-phase catalyst is vastly superior for the production of C₃-C₄ alcohols.

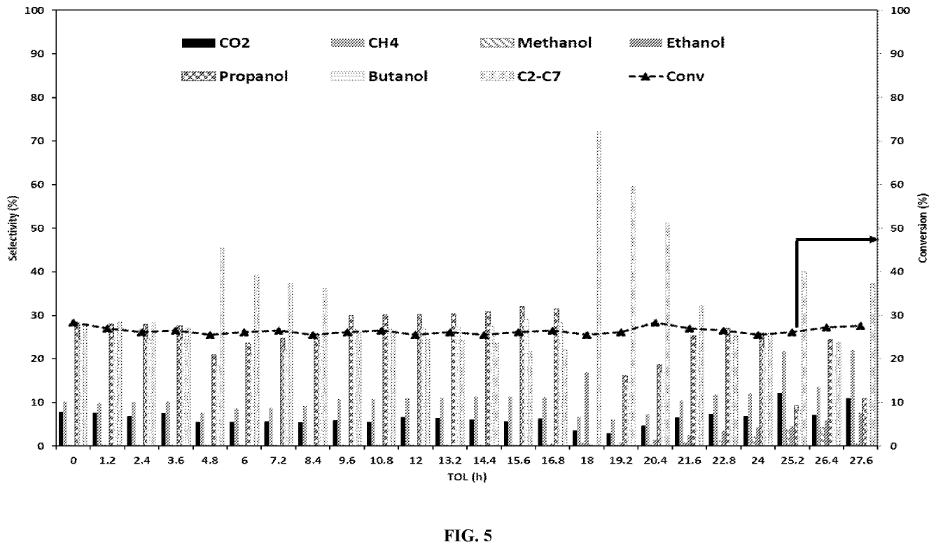

In order to make the catalyst industrially applicable, robust catalytic material must be produced that will endure the harsh conditions provided by fixed bed reactor setups. This goal was achieved by pelleting the catalyst before calcination (in hydrated form). The catalyst (Example 3) was purple in color and successfully retained the β-CoMoO₄ phase. The results were examined for three batches (FIGS. 5-7). When β-CoMoO₄ was pelleted before calcination, it retained high selectivity for C₃-C₄ alcohols. Cumulative selectivity towards C₃-C₄ alcohols was in the range of 50-60%, however, butanol selectivity was higher for β-pellets (Example 3, FIGS. 5-7) than for β-powders (Example 1, FIGS. 1-2). Syngas conversion for β-pellets and β-powders was similar, with conversion amounts at approximately 30%.

It is evident from the data provided herein that the β-CoMoO₄ provides higher selectivity towards C₃-C₄ alcohols, whereas α-CoMoO₄ catalyst produces more methanol and CO₂. Upon further extending the process to dehydration, metal doped heteropoly acids like silicotungstic acid doped with cesium supported on alumina or silica may be used to produce propylene and butylene in high yields.

In the context of the present invention, embodiments 1-16 are described. Embodiment 1 is a calcined composition. The composition includes β-CoₓMo_{y}O_{z}, wherein x ranges from 0.5 to 2.0, y ranges from 0.5 to 2.0, and z ranges from 3.5 to 4.5, wherein said calcined composition is essentially free of catalytically-active amounts of beta-molybdenum carbide (β-Mo₂C), and wherein said calcined composition is essentially free of catalyst-promoting amounts of an alkaline metal promoter or alkaline earth metal promoter. Embodiment 2 is the calcined composition of embodiment 1, wherein the composition exhibits a synthesis gas conversion of at least 10%. Embodiment 3 is the calcined composition of either of embodiments 1 or 2, wherein the composition exhibits a cumulative C₃-C₄ alcohols selectivity of at least 35%.

Embodiment 4 is a process for conversion of a synthesis gas stream into a product stream containing C₃-C₄ alcohols. The process includes exposing said synthesis gas stream to a calcined composition under conditions suitable to convert at least 10% of the synthesis gas stream with at least 35% selectivity for C₃-C₄ alcohols, wherein said calcined composition includes β-CoₓMo_{y}O_{z}, with x ranging from 0.5 to 2.0, y ranging from 0.5 to 2.0, and z ranging from 3.5 to 4.5, wherein said calcined composition is essentially free of catalytically-active amounts of beta-molybdenum carbide (β-Mo2C), and wherein said calcined composition is essentially free of catalyst-promoting amounts of an alkaline metal promoter or alkaline earth metal promoter. Embodiment 5 is the process of embodiment 4, wherein suitable conditions comprise a reaction pressure ranging from 50 to 100 bar. Embodiment 6 is the process of either of embodiments 4 or 5, wherein suitable reaction conditions comprise a reaction temperature ranging from 150 to 450° C. Embodiment 7 is the process of any of embodiments 4 to 6, wherein suitable reaction conditions comprise a synthesis gas CO:H₂ ratio ranging from 0.8:1 to 1.2:1.

Embodiment 8 is a method for making a β-phase catalyst capable of producing C₃-C₄ alcohols from a synthesis gas stream with at least 25% conversion and at least 50% selectivity. The method includes a) preparing a solution comprising a cobalt salt and a molybdenum salt and collecting a precipitate from the solution; b) drying the precipitate to give a dried precipitate comprising one or more hydrates of cobalt molybdenum oxide; c) pelleting the dried precipitate to produce pellets; and d) calcining the pellets to generate the 3-phase catalyst, wherein the pellets are not subjected to mechanical deformation subsequent to calcination. Embodiment 9 is the method of embodiment 8, wherein the cobalt salt is cobalt acetate. Embodiment 10 is the method of either of embodiments 8 or 9, wherein the molybdenum salt is ammonium heptamolybdate. Embodiment 11 is the method of any of embodiments 8 to 10, wherein the solution containing a cobalt salt and a molybdenum salt includes a binary solvent. Embodiment 12 is the method of embodiment 11, wherein the binary solvent includes preferably from 10 to 30% ethanol and from 70 to 90% water, vol:vol. Embodiment 13 is the method of any of embodiments 8 to 12, wherein the precipitate is dried at a temperature ranging from 70 to 150° C. Embodiment 14 is the method of any of embodiments 8 to 13, wherein the precipitate is dried for a period of time ranging from 2 to 6 hours. Embodiment 15 is the method of any of embodiments 8 to 14, wherein the pellets are calcined at a temperature ranging from 300 to 700° C. Embodiment 16 is the method of any of embodiments 8 to 15, wherein the pellets are calcined for a period of time ranging from 2 to 6 hours.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A calcined composition comprising:
β-CoₓMo_{y}O_{z},
wherein x ranges from 0.5 to 2.0, y ranges from 0.5 to 2.0, and z ranges from 3.5 to 4.5, wherein said calcined composition is essentially free of catalytically-active amounts of beta-molybdenum carbide (β-Mo₂C), and
wherein said calcined composition is essentially free of catalyst-promoting amounts of an alkaline metal promoter or alkaline earth metal promoter.

2. The calcined composition of claim 1, wherein the composition exhibits a synthesis gas conversion of at least 10%.

3. The calcined composition of claim 1, wherein the composition exhibits a cumulative C₃-C₄ alcohols selectivity of at least 35%.

4. A process for conversion of a synthesis gas stream into a product stream comprising C₃-C₄ alcohols, said process comprising:
exposing said synthesis gas stream to a calcined composition under conditions suitable to convert at least 10% of the synthesis gas stream with at least 35% selectivity for C₃-C₄ alcohols,
wherein said calcined composition comprises β-CoₓMo_{y}O_{z}, with x ranging from 0.5 to 2.0, y ranging from 0.5 to 2.0, and z ranging from 3.5 to 4.5,
wherein said calcined composition is essentially free of catalytically-active amounts of beta-molybdenum carbide (β-Mo₂C), and
wherein said calcined composition is essentially free of catalyst-promoting amounts of an alkaline metal promoter or alkaline earth metal promoter.

5. The process of claim 4, wherein suitable conditions comprise a reaction pressure ranging from 50 to 100 bar.

6. The process of claim 4, wherein suitable reaction conditions comprise a reaction temperature ranging from 150 to 450° C.

7. The process of claim 4, wherein suitable reaction conditions comprise a synthesis gas CO:H₂ ratio ranging from 0.8:1 to 1.2:1.

8. A method for making a β-phase catalyst capable of producing C₃-C₄ alcohols from a synthesis gas stream with at least 25% conversion and at least 50% selectivity, the method comprising:
a) preparing a solution comprising a cobalt salt and a molybdenum salt and collecting a precipitate from the solution;
b) drying the precipitate to give a dried precipitate comprising one or more hydrates of cobalt molybdenum oxide;
c) pelleting the dried precipitate to produce pellets; and
d) calcining the pellets to generate the β-phase catalyst,
wherein the pellets are not subjected to mechanical deformation subsequent to calcination.

9. The method of claim 8, wherein the cobalt salt is cobalt acetate.

10. The method of claim 8, wherein the molybdenum salt is ammonium heptamolybdate.

11. The method of claim 8, wherein the solution comprising a cobalt salt and a molybdenum salt comprises a binary solvent.

12. The method of claim 11, wherein the binary solvent comprises preferably from 10 to 30% ethanol and from 70 to 90% water, vol:vol.

13. The method of claim 8, wherein the precipitate is dried at a temperature ranging from 70 to 150° C.

14. The method of claim 8, wherein the precipitate is dried for a period of time ranging from 2 to 6 hours.

15. The method of claim 8, wherein the pellets are calcined at a temperature ranging from 300 to 700° C.

16. The method of claim 8, wherein the pellets are calcined for a period of time ranging from 2 to 6 hours.

17. The calcined composition of claim 2, wherein the composition exhibits a cumulative C₃-C₄ alcohols selectivity of at least 35%.

18. The method of claim 9, wherein the precipitate is dried for a period of time ranging from 2 to 6 hours.

19. The method of claim 9, wherein the pellets are calcined at a temperature ranging from 300 to 700° C.

20. The method of claim 9, wherein the pellets are calcined for a period of time ranging from 2 to 6 hours.
